(19) United States
(12) Patent Application Publication
(10) Pub. No.: US 2026/0035331 A1
(43) Pub. Date: Feb. 5, 2026

(54) METHODS FOR EXTRACTING LEVULINIC ACID USING 2-METHYL TETRAHYDROFURAN

(71) Applicant: Origin Materials Operating, Inc., West Sacramento, CA (US)

(72) Inventors: David Ballow, West Sacramento, CA (US); Dimitri Hirsch-Weil, West Sacramento, CA (US); Brett Hodges, West Sacramento, CA (US); Travis Noah, West Sacramento, CA (US)

(21) Appl. No.: 19/354,899

(22) Filed: Oct. 10, 2025

Related U.S. Application Data
(63) Continuation of application No. PCT/US24/24366, filed on Apr. 12, 2024.
(60) Provisional application No. 63/458,718, filed on Apr. 12, 2023.

(51) Int. Cl. C07C 51/48

(52) U.S. Cl. CPC C07C 51/48

(57) ABSTRACT

Levulinic acid and formic acid are valuable chemical intermediaries present in byproducts of some biomass conversion processes. Described herein are commercially viable processes for extracting levulinic acid and formic acid at high recovery. Under the present approach, levulinic acid and formic acid may be extracted from an aqueous reactor product, such as an acidic brine (e.g., calcium chloride brine) feed from a biomass hydrolysis reaction. If present, mineral acid catalysts may be recovered. Embodiments use solvents such as, e.g., 2-methyl tetrahydrofuran, for extracting levulinic acid and formic acid from the aqueous solution.

### CROSS REFERENCE TO RELATED APPLICATIONS

This application is a continuation of International Application No. PCT/US24/24366, filed Apr. 12, 2024, which claims the benefit of, and priority to U.S. Provisional Application No. 63/458,718, filed Apr. 12, 2023, each of which is hereby incorporated by reference in its entirety.

### FIELD

The present disclosure relates to extracting a mixture of aqueous soluble derivatives including levulinic acid (4-oxopentanoic acid) and formic acid from aqueous brine solutions, in some embodiments at ambient conditions.

### BACKGROUND

The shift from fossil resources to decarbonized materials is creating an abundance of potential sources for useful chemical compounds. Depending on the processes involved, converting biomass to useful materials can generate byproducts or waste streams that include useful chemical compounds. The challenge thus far has been developing commercially viable processes to capture these useful chemical compounds, at a sufficient yield and cost.

Levulinic acid (4-oxopentanoic acid) is one such compound. Various biomass conversion processes generate byproducts containing levulinic acid in solution. Levulinic acid is a keto acid commonly derived through the degradation of cellulose, is a precursor to biofuels and an intermediary for numerous other chemical compounds, resins, plasticizers, etc. However, levulinic acid has a high boiling point, and is difficult to separate from other byproducts and waste streams common in biomass conversion processes.

As such, there are at least a few major issues in producing and/or recovering levulinic acid from biomass. First, levulinic acid is difficult to separate from the mineral acid catalysts (sulfuric acid or HCl) or the byproducts. Second, current processes generally require high temperature reaction conditions, generally long digestion periods of biomass, specialized equipment to withstand hydrolysis conditions, and as a result, the yield of the levulinic acid is quite low (e.g., current processes generate yields of 50 stoichiometric percent or less). At such high capital costs and low yield, the current processes lack commercial viability. Also, the solids obtained under the reaction conditions of these existing processes can result in fouling of the reactor and downstream equipment due to plugging or sticking of the char to the internals of the equipment. Compared to levulinic acid production processes, recovering levulinic acid from biomass conversion waste streams could be a more viable approach, provided that the recovery results in sufficient yield and purity.

Formic acid is another common byproduct of biomass conversion, and also has an abundance of uses as an intermediary in chemical synthesis. Industry has struggled to develop commercially viable processes to separate formic acid a sufficient yield and cost.

What is needed, then, are processes to separate levulinic acid and formic acid produced by biomass conversion processes, at sufficient yields and purities to achieve commercial viability.

### BRIEF SUMMARY

This disclosure relates to the extraction of valuable chemical intermediaries from the byproducts of biomass conversion. Described herein are commercially viable processes for extracting levulinic acid and formic acid at high recovery. Under the present approach, levulinic acid and formic acid may be extracted from an aqueous reactor product, such as an acidic brine (e.g., calcium chloride brine). For example, the aqueous feed may be a feed from a biomass hydrolysis reaction. In some embodiments the extraction may occur before or after mineral acid catalyst recovery. The aqueous phase may be a combination of salt and water, with or without mineral acid. In some embodiments the extraction may proceed under ambient conditions. In some embodiments, 2-methyl tetrahydrofuran may be used as an extraction solvent for levulinic acid and formic acid from calcium chloride brine solutions.

Embodiments of the present approach may take the form of a process for extracting levulinic acid from an aqueous feed having levulinic acid. For example, the aqueous feed may be a calcium chloride brine containing levulinic acid, such as a brine byproduct from another process. It should be appreciated that the embodiments are described in terms of a continuous process, but that the present approach may be adapted into a batch process if desired. The aqueous feed having levulinic acid is introduced to a first separation column, and a solvent feed is introduced to the first separation column countercurrent to the aqueous feed. The solvent may comprise 2-methyl tetrahydrofuran. It should be appreciated that a variety of separation columns may be suitable for use in the present approach. For extraction, and in particular liquid-liquid extraction, the first separation column may be, for example, a commercially agitated column, a packed column, or a trayed column. In such extraction columns, the aqueous feed is introduced to a portion of a first end of the first separation column, and the solvent feed is introduced to a portion of a second end of the first separation column, the second end opposite of the first end. The precise inlet (e.g., tray or column height) will depend on the particular embodiment and can vary depending on the particular aqueous feed and solvent stream. A rich solvent stream comprising solvent and extracted levulinic acid, and an aqueous raffinate, are extracted from the first separation column. The rich solvent stream may be introduced to a second separation column, to separate the levulinic acid from the solvent. The second separation column is, preferably, a distillation column. In the disclosed embodiments, the distillation column operates at a pressure between 1 bar abs and 2 bar abs, and a temperature between 80° C. and 220° C. The solvent and the levulinic acid may be removed from the second separation column as separated streams, i.e., distillate and bottoms, respectively. At least a portion of the solvent feed may comprise the recycled extracted solvent feed from the second separation column.

In some embodiments, the aqueous feed comprises a mineral acid. The mineral acid may be, for example, hydrochloric acid, nitric acid, phosphoric acid, sulfuric acid, boric acid, hydrofluoric acid, hydrobromic acid, perchloric acid, or hydroiodic acid. Some embodiments may include recovering the mineral acid. For example, the mineral acid recovery may occur prior to removing levulinic acid from the second separation column, or after removing levulinic acid from the second separation column. In some embodiments, the process may further include separating formic acid from levulinic acid

### DESCRIPTION OF THE DRAWINGS

FIG. 1 illustrates a demonstrative embodiment of a process for extracting levulinic acid and formic acid from calcium chloride brine solutions, according to the present approach.

FIG. 2 is a schematic showing a prototype embodiment of a process for extracting levulinic acid and formic acid from calcium chloride brine solutions, according to the present approach.

### DESCRIPTION

The following description is of the best currently contemplated mode of carrying out exemplary embodiments of the present approach. The description is not to be taken in a limiting sense, and is made merely for the purpose of illustrating the general principles described herein.

Disclosed herein are processes for removing target components, and in particular levulinic acid and formic acid, from an aqueous brine solution, such as a calcium chloride brine solution. It should be appreciated that the brine may be comprised of a salt and a mineral acid. The salt may be selected from, among others, calcium chloride, lithium chloride, magnesium chloride, sodium chloride, potassium chloride, and combinations thereof. The mineral acid can be, for example, hydrochloric acid, nitric acid, phosphoric acid, sulfuric acid, boric acid, hydrofluoric acid, hydrobromic acid, perchloric acid, and hydroiodic acid. In an example of a preferred embodiment, the salt is calcium chloride in water at a range of 5 wt. % to 60 wt. %, with or without a mineral acid.

The present approach involves the use of a solvent for liquid-liquid extraction of the target compounds from an aqueous brine. Example solvents include, but are not limited to ortho-Cresol, 2-methyl tetrahydrofuran, 2,5-dimethyltetrahydrofuran, and gamma-Valerolactone. In preferred embodiments, 2-methyl tetrahydrofuran (“m-THF”) is used as the extraction solvent to remove levulinic acid and formic acid from an acidic calcium chloride brine solution. The m-THF is contacted in a countercurrent column with the acidic brine to extract the target components. The rich m-THF loaded with target components is then recovered via distillation, leaving the higher boiling levulinic acid as a column bottoms product.

Solvent types include alcohols, ethers, nitriles, ketones, and esters. Alcohol solvents useful in the present approach may be selected from, e.g., alkyls and aryls, and may be branched or linear chain species. Preferred alcohols include, but are not limited to, propanol, n-butanol, tert-butanol, benzyl alcohol, and ortho-Cresol. Ether solvents useful in the present approach may be selected from, e.g., alkyls, aryls, and cyclic ethers, and may be branched or linear chain species. Preferred ethers include, but are not limited to, methyl-tertbutyl ether, 2-methyltetrahydrofuran, and 2,5-dimethyltetrahydrofuran. Nitrile solvents useful in the present approach may be selected from, e.g., alkyls and aryls, and may be branched or linear chain species. A preferred nitrile solvent is acetonitrile. Ketone solvents useful in the present approach may be selected from, e.g., alkyls and aryls, and may be branched or linear chain species. A preferred ketone solvent is methyl isobutyl ketone. Ester solvents useful in the present approach may be selected from, e.g., alkyls, aryls, and cyclic esters, and may be branched or linear chain species. Preferred esters include, but are not limited to, ethyl acetate and gamma-Valerolactone.

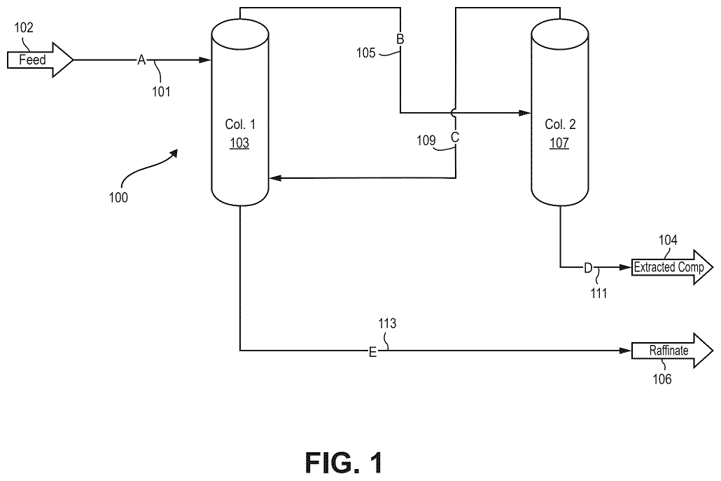

FIG. 1 illustrates an embodiment of a process according to the present approach. Feed (A) enters the top of an extraction column (Col. 1) and flows downward at moderate temperatures (5-130° C.) and pressures (1-10 bar-abs). The extraction column (Col. 1) is designed to create close contact between immiscible phases. Such separation columns are commonly available in the chemical processing industry and known by those having an ordinary level of skill in the art. Examples include commercially agitated columns, packed columns, and trayed columns. Lean solvent (C) enters the bottom of the column and flows upward. The countercurrent arrangement results in a rich solvent (B) exiting the top of the extraction column (Col. 1), and an extracted brine raffinate (E) leaving the bottom of the extraction column.

In the illustrated embodiment, rich solvent (B) flows to a distillation column (Col. 2) operating at pressures between 1 and 2 bar-abs and temperatures in the range of 80° C. and 220° C. The distillation recovers the m-THF as lean solvent (C) overhead. That lean solvent (C) is returned to the extraction column (Col. 1). The remaining material in the bottom of the distillation column (Col. 2) is the target extracted components (D).

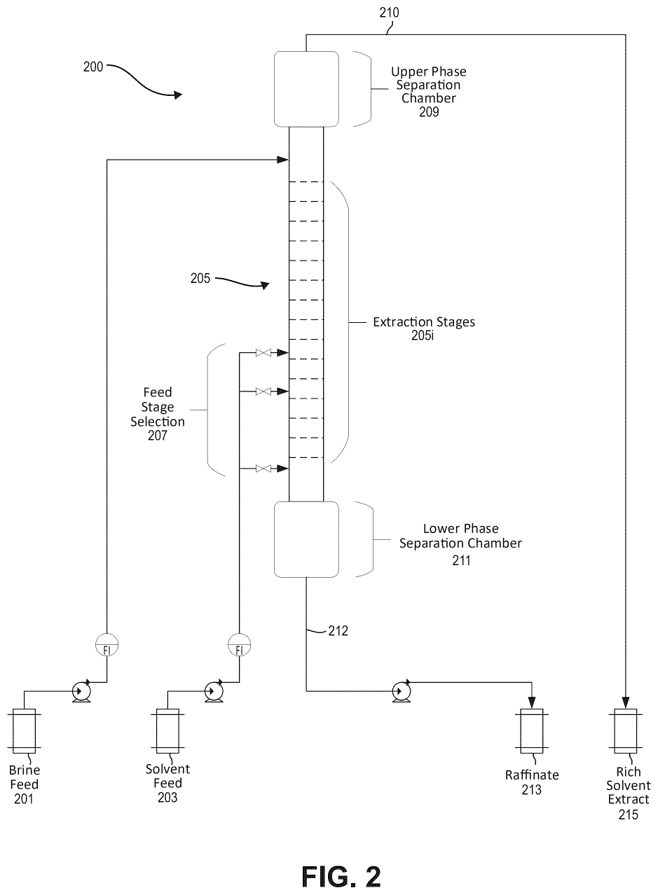

FIG. 2 is a schematic showing a prototype embodiment of a process for extracting levulinic acid and formic acid from calcium chloride brine solutions, according to the present approach. In this embodiment, the brine feed heavy phase and m-THD solvent light phase are pumped to a multi-extraction stage agitated column. Rich solvent extract is recovered at the top of column from an upper phase separation chamber, and the aqueous raffinate is recovered from the bottom of column through a lower phase separation chamber. It should be appreciated that the feeds may be located at different stages, and that the optimum stage for a particular embodiment may be determined through routine skill in the art. It should be appreciated that embodiments may include temperature and/or flow sensors and at one or more locations. The prototype process described herein is configured to extract levulinic and formic acids from an acidic brine solution using m-THF as the solvent. In the initial trials, the aqueous feed contained approximately 1.1 wt % levulinic acid and 0.6 wt % formic acid. The prototype apparatus included a 3″ diameter×60-stage glass commercially agitated column with Alloy C276. All stages were equipped with standard size impellers.

Pilot trial runs were performed at temperatures slightly above ambient conditions in the range of 20° C. to 30° C. Temperatures were controlled across the column using electrical heat tape to establish a consistent temperature profile. Solvent and brine were metered into the column using standard pumps and standard mass flow meters. Mass flow measurement and careful metering allowed the trial to test varying solvent to brine flow ratios. The system was operated to result in a continuous brine phase with solvent dispersed throughout by the column internals. Tubing runs and valving were used to select the feed location of the solvent allowing the trial to vary number of actual column stages for extraction. Column effluent stream measurements were achieved using timed volumetric measurements by filling a sample container over a prescribed, measured amount of time and recording the volume obtained. It should be appreciated that column configurations may vary in other embodiments, to achieve acceptable recovery for the given embodiment. These variations are routine in the art.

The column was preloaded with freshly made 21% by weight calcium chloride brine solution prior to each trial run. Feed brine was supplied from prior Origin batch reactor runs at the West Sacramento facility. The feed and solvent were fed directly from their respective drums. The following procedure was used:
The column was filled with clean CaCl₂) solution to the feed inlet.
Column agitation was started.
The feed and solvent were started to the column at the desired rates.
The phase interface was observed and controlled to the approximate mid-point of the upper disengaging chamber by starting the bottoms take-off pump.
The column was allowed to reach a steady state, approximately five (5) turnovers from initial startup, before sampling the raffinate and extract phases. Extract and raffinate flows were measured as a part of the sampling process.

Raffinate samples were analyzed using gas chromatography and HPLC. In prototype processes, levulinic acid recoveries in the range of 73% to 99.5% (measured as levulinic acid removed from brine) were demonstrated in trial runs of the embodiment described above, at solvent to feed ratios of 0.2 to 0.60. Recovery of m-THF and the purity of levulinic acid are being analyzed. It should be appreciated that the extraction efficiency of the present approach may, depending on the embodiment and the aqueous feed, fall in the range of 70% to 99.5%,

The terminology used herein is for the purpose of describing particular embodiments only and is not intended to be limiting of the approach. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The present approach may be embodied in other specific forms without departing from the spirit or essential characteristics thereof. The present embodiments are therefore to be considered in all respects as illustrative and not restrictive, the scope of the invention being indicated by the claims of the application rather than by the foregoing description, and all changes which come within the meaning and range of equivalency of the claims are therefore intended to be embraced therein.

What is claimed is:

1. A process for extracting levulinic acid from an aqueous feed having levulinic acid, the process comprising:
introducing the aqueous feed that comprises a saltwater solution to a first separation column, wherein the saltwater solution comprises the levulinic acid and at least one member selected from the group consisting of calcium chloride, lithium chloride, magnesium chloride, sodium chloride, and potassium chloride;
introducing a solvent feed to the first separation column countercurrent to the aqueous feed;
removing a rich solvent stream from the first separation column, the rich solvent stream comprising solvent and extracted levulinic acid;
removing an aqueous raffinate from the first separation column;
introducing the rich solvent stream to a second separation column;
removing the solvent from the rich solvent stream in the second separation column;
removing the solvent feed from the second separation column; and
removing levulinic acid from the second separation column.

2. The process of claim 1, wherein the saltwater solution comprises the calcium chlorine.

3. The process of claim 1, wherein the solvent is selected from the group consisting of propanol, n-butanol, tert-butanol, benzyl alcohol, ortho-Cresol, methyl-tertbutyl ether, 2-methyltetrahydrofuran, 2,5-dimethyltetrahydrofuran, acetonitrile, methyl isobutyl ketone, ethyl acetate and gamma-Valerolactone.

4. The process of claim 1, wherein the solvent comprises 2-methyl tetrahydrofuran.

5. The process of claim 1, wherein the first separation column is selected from the group consisting of an agitated column, a packed column, and a trayed column.

6. The process of claim 1, wherein the second separation column is a distillation column.

7. The process of claim 6, wherein the distillation column operates at a pressure of 1-2 bar-abs, and a temperature between 80° C. and 220° C.

8. The process of claim 1, wherein the aqueous feed is introduced to a portion of a first end of the first separation column, and the solvent feed is introduced to a portion of a second end of the first separation column, the second end opposite of the first end.

9. The process of claim 1, wherein at least a portion of the solvent feed comprises the extracted solvent feed from the second separation column.

10. The process of claim 1, wherein the levulinic acid extracted from the second separation column is recovered in an amount of from 73% to 99.5% recovery.

11. The process of claim 1, wherein the aqueous feed further comprises formic acid, and the process further comprises removing formic acid from the second separation column.

12. The process of claim 1, wherein the aqueous feed further comprises a feed from a biomass hydrolysis reaction.

13. The process of claim 1, wherein the aqueous feed further comprises a mineral acid.

14. The process of claim 13, wherein the mineral acid is selected from the group consisting of hydrochloric acid, nitric acid, phosphoric acid, sulfuric acid, boric acid, hydrofluoric acid, hydrobromic acid, perchloric acid, and hydroiodic acid.

15. The process of claim 13, further comprising recovering the mineral acid.

16. The process of claim 15, wherein the recovering of the mineral acid occurs prior to removing levulinic acid from the second separation column.

17. The process of claim 15, wherein the recovering of the mineral acid occurs after removing levulinic acid from the second separation column.

18. The process of claim 1, wherein the solvent is an alcohol, an ether, a nitrile, a ketone, or an ester.

19. The process of claim 1, wherein the aqueous feed includes formic acid, and the process further comprises separating formic acid from levulinic acid.
